(19) United States
(12) Patent Application Publication
(10) Pub. No.: US 2002/0098172 A1
(43) Pub. Date: Jul. 25, 2002

(54) Coenzyme Q10 formulation and process methodology for soft gel capsules manufacturing

(76) Inventors: Ronald G. Udell, Beverly Hills, CA (US); Siva P. Hari, Riverside, CA (US)

Correspondence Address: KAMRAN FATTAHI, KELLY BAUERSFELD LOWRY AND KELLEY, LLP, 6320 CONAGO AVENUE, SUITE 1650, WOODLAND HILLS, CA, 91367, US

(21) Appl. No.: 09/873,156

(22) Filed: Jun. 1, 2001

Related U.S. Application Data
(60) Provisional application No. 60/263,953, filed on Jan. 24, 2001.

(51) Int. Cl. A61K 38/43; A61K 35/78; A61K 31/355

(52) U.S. Cl. 424/94.1; 424/757; 514/458

(57) ABSTRACT

A process and formulation for a soft gel capsule that results in at least half the normal daily requirement of Coenzyme Q₁₀ being absorbed by a healthy human from a capsule having a reduced amount of Coenzyme Q₁₀, GelOil SC, Vitamin E and an optional additional antioxidant.

### FIELD OF THE INVENTION

This invention relates to an improved formulation and process methodology of Coenzyme Q₁₀ for producing soft gel capsules with improved absorption.

### BACKGROUND OF THE INVENTION

Coenzyme Q₁₀ (CoQ₁₀ or Ubiquinone) is a large molecular weight (863.63 grams) lipid compound that is produced in the liver and perhaps other body organs. The total human body content is estimated to be 1.4 to 1.8 grams, depending on the age and the physical fitness of the individual. Although CoQ₁₀ is found in the mitochondria and other organelles of every living cell, it appears to be most abundant in tissues with a high number of mitochondria and a high level of metabolic activity. For example, in the metabolically inactive blood there is approximately 4 mg, in the heart, and in the skeletal muscle 1000 mg. The blood acts as a CoQ₁₀ reservoir and transport media between endogenous CoQ₁₀ synthesis in the liver, exogenous CoQ₁₀ absorption from digested food substances in the intestinal tract, and the body cells. Endogenous synthesis appears to be responsible for 56 percent and exogenous sources for 44 percent of the body is CoQ₁₀, requirements. These numbers are currently being studied and endogenous CoQ₁₀ synthesis may be significantly deficient in the elderly. Furthermore, certain disease states, such as mitochondrial myopathy, and prescription drugs, such as cholesterol-lowering statin drugs, seem to deplete the endogenous CoQ₁₀ levels in the body. These deficiencies are not related to the total caloric intake, but rather to the vitamin content of ingested foods as the body requires multiple vitamins for the synthesis of CoQ₁₀.

CoQ₁₀ requirements of the body are also variable between individuals and are dependent on age, physical activity, and disease. It is estimated that the body CoQ₁₀ utilization is between 5 and 9 mg per day. Intercellular CoQ₁₀ is required for the synthesis of energy and therefore essential for life. Energy synthesis occurs in the mitochondria, where CoQ₁₀ provides an electron for the electron transport chain in the cytochrome system, in which adenosine tripohosphate (ATP) is synthesized. As CoQ₁₀ gives up an electron for ATP synthesis, it gets oxidized. If CoQ₁₀ is used as an antioxidant, it gets oxidized and is no longer available to provide electrons and function in the synthesis of ATP. Under conditions of high metabolic stress, endogenous sources may become inadequate to meet the body is CoQ₁₀ requirement for ATP synthesis. Under such conditions, dietary CoQ₁₀ supplementation has been shown to be an effective source. An improved soft gel formulation and process of CoQ₁₀ soft gel capsule manufacturing has used to treat heart failure, chronic fatigue and patients with psoriasis and planter warts. In all cases, it has been found that the improved soft gel formulation, at doses of 30-100 mg/day of CoQ₁₀, have been proven to be superior to commercially available 60 mg dry powder capsules, and existing 100 mg/day CoQ₁₀ soft gel formulations.

An appropriate CoQ₁₀ dosage for a normal individual compared to the dosage necessary for a diseased individual has been difficult to ascertain. Recommended doses of 10 to 30 mg/day were found to be ineffective for patients with significant CoQ₁₀ deficiencies. In the past 15 years, it has become generally accepted that poor intestinal absorption of certain CoQ₁₀ formulations limits their effective use. For this reason, 50 and 150 or even 200 mg tablets or capsules are commercially available to the consumer, at a considerable higher cost, the main cost driver being the CoQ₁₀.

Folkers et al. (U.S. Pat. No. 4,824,669) addresses a soft gel capsule with CoQ₁₀ and at least one vegetable oil. This formulation was determined to increase blood CoQ₁₀ levels to 2.5 μg/ml compared to 1.6 μg/ml for an equivalent 100 mg dose of dry powder CoQ₁₀. Many different CoQ₁₀ formulations have appeared which are claimed to increase intestinal absorption. However, intestinal absorption data, collected under near basal conditions, which compare CoQ₁₀ alone in oil with dry powder CoQ₁₀, are inconclusive.

### SUMMARY OF THE INVENTION

The present invention comprises a stable and nontoxic soft gel Coenzyme Q₁₀ formulation and process methodology of Coenzyme Q₁₀ for increased Coenzyme Q₁₀ absorption levels in the human intestinal tract. A preferred soft gel formulation includes Coenzyme Q₁₀ Gel Oil SC and Vitamin E (mixed tocopherols) added as a functional antioxidant. An additional ingredient, an antioxidant, may be added to this formula for additional antioxidant benefits. The preferred soft gel Coenzyme Q₁₀ formulation is administered twice a day in dosages of about 30 mg, thereby reducing the Coenzyme Q₁₀ cost while producing the desired retained Coenzyme Q₁₀, in the human body.

It is therefore an objective of the present invention to provide an improved soft gel formulation of CoQ₁₀ and a methodology of formulation processing that produce a significantly greater bioavailability of CoQ₁₀, than existing soft or dry formulations. To this end, the present formulation contains CoQ₁₀ and refined soybean oil along with wetting and suspending agents derived from vegetables (GelOil SC) to improve the solubility of CoQ₁₀. The composition of GelOil SC includes refined soybean oil (CAS# 8001-22-7), glycerides (mono-, di- and tri-glycerides of 16 to 18 carbon chain length) (CAS# 91052-54-9) and polyglycerol oleate (CAS# 9007-48-1). An additional antioxidant, either from natural or synthetic sources, can be added in order to prepare a potent combination antioxidant formulation.

It is a further objective of the present invention to provide a soft gel formulation of CoQ₁₀ and methodology of administration that produces greater absorption in the intestine.

### DETAILED DESCRIPTION OF THE PREFERRED EMBODIMENTS

The unique formulation involves the following sequence of ingredients and process methodology.

Heat GelOil SC to 25 to 35° C.;

Simultaneously add in a container under vacuum the following ingredients to the heated GelOil SC:

Coenzyme Q₁₀ Vitamin E, and if desired additional antioxidant in compatible form, the vacuum being to prevent oxidation of any of the ingredients;

Blend and continuously stir of all the ingredients into a mixture;

Cool the mixture to 25 to 30° C.;

Mix the mixture within the container under a blanket of nitrogen gas to prevent oxidation of any of the ingredients; and

Encapsulate the mixture in a soft gel capsule. If the cooled mixture sits for any length of time under its blanket of nitrogen before encapsulation, re-mix under the blanket of nitrogen to assure a homogeneous mixture for encapsulation.

Typical amounts per capsule are:

50 to 500 mg of GelOil SC;

30 to 100 mg of Coenzyme Q₁₀;

10-100 IU Vitamin E; and if desired

0.5 to 500 mg of additional antioxidant.

The bioavailability or intestinal absorption of CoQ₁₀ has been a major controversy in the international CoQ₁₀ research community. Previous data indicate that only 1 to 3% of a dry powder CoQ₁₀ formulation is absorbed through the lacteals in the intestines and appears in the blood over a twelve hour interval. In general, blood levels of 1.2 to 1.6 μg/ml have been reported, when taking 30 to 60 mg/day dry powder CoQ₁₀ formulation for 30 days. It has been reported that when a dry powder CoQ₁₀ formulation is taken with a fat, such as peanut butter, steady-state blood levels of 2.0 to 2.8 μg/ml are measurable. Multiple clinical trials were conducted in the United States and Europe using the Folkers (U.S. Pat. No. 4,824,669) soft gel. With a dosage of 100 mg/day multiple investigators have reported group mean blood levels of 2.3 to 3.5 μg/ml depending on the laboratory conducting the measurement.

The present 30 mg CoQ₁₀ soft gel formulation of CoQ₁₀ provides approximately 50%, and with two capsules 100%, of the daily CoQ₁₀ requirements of a normal sedentary individual. It would take at least three of the dry powder 30 mg CoQ₁₀ capsules to produce the same effects as one of the present invention in 30 mg soft gel form, and six of the dry powder 30 mg CoQ₁₀ capsules to produce the same effect as two of the present 30 mg CoQ₁₀ soft gel capsules. Regardless of the absorption mechanism, the significantly higher basal blood CoQ₁₀ levels (167%) and the 273% greater absorption rate found in studies, establish that the present soft gel formulation is indeed a superior product to the dry CoQ₁₀ formulations. This may be especially true for those individuals whose daily CoQ₁₀ requirement is elevated due to: high physical activity; a need for CoQ₁₀ as an antioxidant; or active disease associated with known CoQ₁₀ deficiencies.

Cellular CoQ₁₀ content is a function of the number and quality of the cellular mitochondria. For example, the failing heart muscle has 2.2 μg CoQ₁₀ per mg tissue and a blood CoQ₁₀ deficiency (0.3-0.5 μg/ml). The normal conditioned heart has 6.3 μg/gm in its tissue, and a low basal blood level (0.5-0.6 μg/ml). These results indicate that supplemental CoQ₁₀ enters the cell. This observation has also been reported for skeletal muscles of trained and non-trained athletes.

The subjective and objective responses to supplemental CoQ₁₀ in the normal individual appear more rapidly compared to that of the physically unfit or the diseased individual with a CoQ₁₀ deficiency. The most probable reason for this observation is that the metabolic machinery (mitochondria) is viable in the non-diseased normal volunteer, whereas the mitochondria are atrophied in the cells of de-conditioned and diseased individuals. Therefore, it takes time in the diseased individual to build up the mitochondria to a more normal activity level and to normalize their distribution in the organ system involved.

Thus there has been described a novel CoQ₁₀ formulation and method of formulation, which fulfill all the objects and advantages sought therefor. Many changes, modifications, variations and applications of the subject invention will become apparent to those skilled in the art after consideration of the specification.

All such changes, modifications, alterations and other uses and applications which do not depart from the spirit and scope of the invention are deemed to be covered by the invention which is limited only by the claims that follow:

1. A human treatment method to improved absorption of Coenzyme Q₁₀ into an intestinal tract and to maintain of Coenzyme Q₁₀ basal blood levels comprising:
administration of a soft gel formulation of 30-100 mg/day of Coenzyme Q₁₀, GelOil SC, and Vitamin E.

2. The method as defined in claim 1 wherein said soft gel formulation further includes:
an additional antioxidant.

3. A soft gel capsule formulation for improved absorption of Coenzyme Q₁₀ into a human intestinal tract of Coenzyme Q₁₀ including in each capsule:
30 to 100 mg Coenzyme Q₁₀;
50 to 500 mg GelOil SC; and
10-100 IU Vitamin E.

4. The soft gel capsule formulation as defined in claim 3 further including:
0.5 to 500 mg of additional antioxidant.

5. A process to manufacture soft gel capsules containing an improved formulation of Coenzyme Q₁₀ said process per capsule including:
heating 50 to 500 mg of refined soybean oil, mono-, di- and tri-glycerides of 16 to 18 carbon chain length and polyglycerol oleate to 30 to 35° C.;
blending in a container under vacuum the heated refined soybean oil, mono-, di- and tri-glycerides of 16 to 18 carbon chain length and polyglycerol oleate, 30 to 100 mg of Coenzyme Q₁₀, and 10 to 100 IU Vitamin E;
cooling the blended mixture to at least 30° C.;
mixing the cooled mixture under a nitrogen blanket; and
encapsulating the mixed, cooled mixture in a soft gel capsule.

6. The process as defined in claim 5 wherein said blending further includes:
adding 0.5 to 500 mg of an additional antioxidant.

7. The process as defined in claim 5 wherein the refined soybean oil, mono-, di- and tri-glycerides of 16 to 18 carbon chain length and polyglycerol oleate is GelOil SC.

8. The process as defined in claim 5 wherein said mixing further includes:
storing the cooled mixture under a nitrogen blanket; and
remixing the cooled stored mixture under a nitrogen blanket.
